(19) United States
(12) Patent Application Publication
(10) Pub. No.: US 2007/0066894 A1
(43) Pub. Date: Mar. 22, 2007

(54) Remote wireless control device for an ultrasound machine and method

(75) Inventors: Kris M. Bartol, Nashua, NY (US); Stanley J. Graziano, Atkinson, NH (US); William H. Kelton, Manchester, NH (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., BA Eindhoven (NL)

(21) Appl. No.: 10/550,046

(22) PCT Filed: Mar. 25, 2004
(86) PCT No.: PCT/IB04/01264

§ 371 Date: Sep. 21, 2005

Related U.S. Application Data
(60) Provisional application No. 60/458,795, filed on Mar. 28, 2003.

(51) Int. Cl. A61B 8/00

(52) U.S. Cl. 600/437

(57) ABSTRACT

Ultrasound machine enabling remote wireless control and including a user-interface input section having controls, an ultrasonic transducer for transmitting and receiving waves, a screen for displaying an image and a control unit coupled to the input section, screen and transducer to enable control of the transducer, processing of received waves and images displayed on the screen via the controls of the input section. A remote control device is wirelessly coupled to the control unit and includes only a portion of the controls of the input section to enable remote control of the transducer, processing of the received waves and images displayed on the screen. The controls on the remote control device are typically the most frequently used controls. The remote control device is positionable in any position in which it is comfortable for the sonographer to operate the controls, thereby providing an ergonomic benefit.

The present invention relates generally to an ultrasound machine having a remote, wireless control device which enables ergonomic placement of certain controls of the ultrasound machine and remote adjustment of the controls of the ultrasound machine, and more particularly to a remote control device for an ultrasound machine which includes a subset of the controls for the ultrasound machine and is wirelessly coupled to the ultrasound machine.

The present invention also relates to a method for ergonomically placing the controls of an ultrasound machine using a remote, wireless control device.

Modern ultrasound diagnostic systems, also referred to as ultrasound machines herein, include numerous controls for adjusting the transmission of the ultrasonic waves, the reception of the ultrasonic waves, the processing of the ultrasonic waves to form images and the manipulation of the images to extract pertinent data. Manipulation of the images often involves the use of a positioning device to position a cursor or indicator for delimiting an area of interest about which information is desired to be obtained.

These controls take the form of sliders, selectors, knobs, switches, buttons, trackballs, touch pads and the like which are integrated into a control panel formed on the ultrasound machine. For example, some controls are mode buttons, a positioning adjustment trackball, a depth knob, a gain knob and a focus knob. In view of the presence of numerous controls on the control panel, the control panel is often quite large and complex and problems may be encountered in finding a desired control for adjustment during the ultrasonic examination. Adjusting an incorrect control will result in a delay in the examination and the possibility of having to wait until the same examination conditions are created to obtain the desired images.

U.S. Pat. No. 6,135,958 (Mikula-Curtis et al.) describes an ultrasound imaging system with a touch-pad pointing device used to perform measuring, tracing, navigating, caliper placing, annotating and menu selection of images or objects in the images displayed on a screen. The touch-pad pointing device can be coupled to the ultrasound imaging system via wireless connection using, for example, infrared, radio-frequency or audible signals. Use of the touch-pad pointing device enables adjustment of only the position of a pointer on the image displayed on the screen. As such, it is a disadvantage that adjustment of the transmission and reception of the ultrasonic waves by a transducer, processing of the ultrasonic waves and the actual images displayed on the screen cannot be performed without accessing the keyboard or controls on the user-interface input section arranged on the housing of the ultrasound imaging system.

It is an object of the present invention to provide a new ultrasound machine in which certain controls are ergonomically placed so that the sonographer can position these controls in a position in which they are most comfortable for the sonographer, including locations separated from the ultrasound machine itself.

It is another object of the present invention to provide a new ultrasound machine having a remote, wireless control device which enables remote adjustment of several controls of the ultrasound machine including controls which enable adjustment of the transmission and reception of the ultrasonic waves by a transducer, processing of the ultrasonic waves and the images displayed on a screen of the ultrasound machine.

It is still another object of the present invention to provide a new remote control device for an ultrasound machine which includes a subset of the controls for the ultrasound machine and is wirelessly coupled to the ultrasound machine. The subset of controls may be the most frequently used controls.

In order to achieve these objects and others, an ultrasound machine in accordance with the invention includes a user-interface assembly including a user-interface input section having a plurality of controls, an ultrasonic transducer for transmitting and receiving ultrasonic waves, a screen for displaying an ultrasound image, a control unit coupled to the user-interface assembly, the screen and the transducer to enable control of the transducer, control of the processing of the received ultrasonic waves and control of the images displayed on the screen via the controls of the user-interface input section and a remote control device wirelessly coupled to the control unit.

It is a feature of the invention that the remote control device includes only a portion of the controls of the user-interface input section, i.e., a subset thereof, to enable remote control of the transducer, of the processing of the received ultrasonic waves and of the images displayed on the screen. The controls on the remote control device are typically the predetermined, most frequently used controls and typically include at least one control for adjustment of the transmission and reception of the ultrasonic waves by the transducer, adjustment of the processing of the ultrasonic waves and adjustment of the images displayed on the screen. As such, easy access to the most frequently used controls is provided by the remote control device without the need to search for a particular control in the user-interface input section which is typically crowded with numerous controls.

An important advantage obtained by the invention is that the remote control device can be placed by the sonographer in any position in which it is comfortable for the sonographer to operate the controls, thereby providing an ergonomic benefit, yet still enable the sonographer to access essentially all of the controls needed for a given examination. Generally, only some of the controls are used for every examination, which controls would be situated on the remote control device, and other controls are used infrequently, which controls would not be situated on the remote control device and thus would require the sonographer to access the user-interface input section.

In one embodiment, the remote control device includes a touch screen programmable to display the controls. In addition to one or more control for adjusting the transmission, reception and/or processing of the waves and/or the images on the screen, the touch screen may be programmed to display a pointing region for enabling positional adjustment of an indicator on the screen. In the alternative, a trackball may be arranged on the remote control device for enabling positional adjustment of the indicator on the screen.

The remote control device typically includes an attachment mechanism for attaching it to an object such as a bed rail. One attachment mechanism includes a pair of clamps, springs for coupling the clamps to one another and a handle for moving one clamp toward the other. Other attachment mechanisms may also be used.

A method for controlling an ultrasound machine in accordance with the invention entails transmitting and receiving ultrasonic waves via an ultrasonic transducer, processing the received ultrasonic waves to generate an image, displaying the image generated from the received ultrasonic waves on a screen, providing a user-interface assembly including a user-interface input section having a plurality of controls for enabling control of the transmission, reception and processing of the ultrasonic waves and images displayed on the screen and arranging only a portion of the controls of the user-interface input section on a remote control device. The transmission, reception and processing of the ultrasonic waves and images displayed on the screen are wirelessly controlled via the controls on the remote control device to enable the sonographer to place the remote control device in an ergonomically pleasing, i.e., comfortable, position. The same enhancements described above for the ultrasound machine in accordance with the invention may be applied in this method as well.

The invention, together with further objects and advantages hereof, may best be understood by reference to the following description taken in conjunction with the accompanying drawings, wherein like reference numerals identify like elements and wherein:

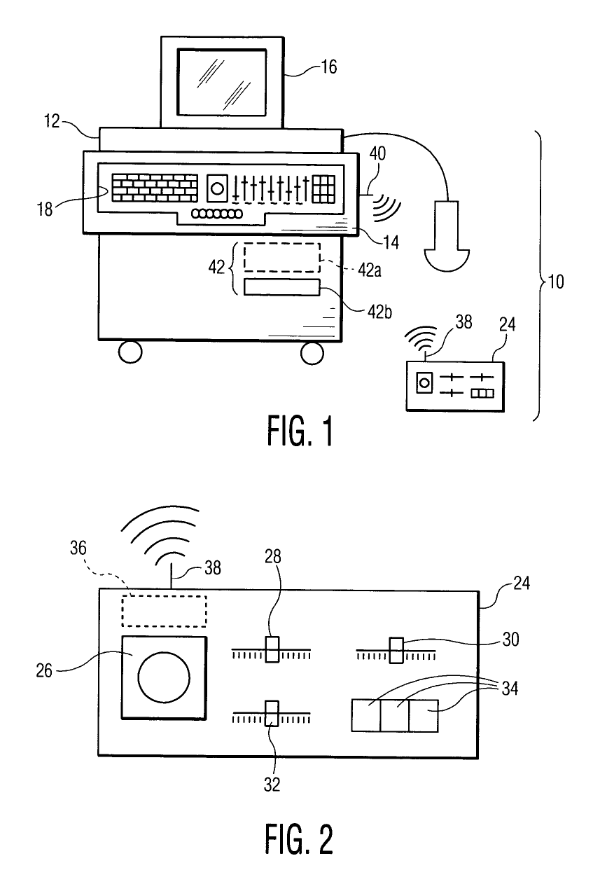

FIG. 1 is an illustration of an ultrasound machine including a remote control device in accordance with the invention;

FIG. 2 is an enlarged view of the remote control device shown in FIG. 1;

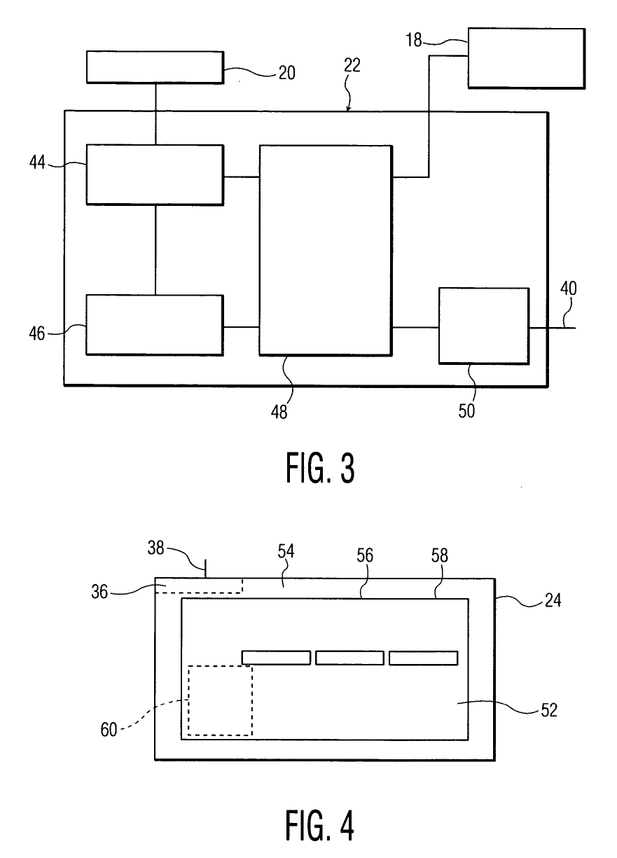

FIG. 3 is a schematic of the electrical connection in the ultrasound machine shown in FIG. 1;

FIG. 4 is a view of another embodiment of a remote control device for use with the ultrasound machine shown in FIG. 1;

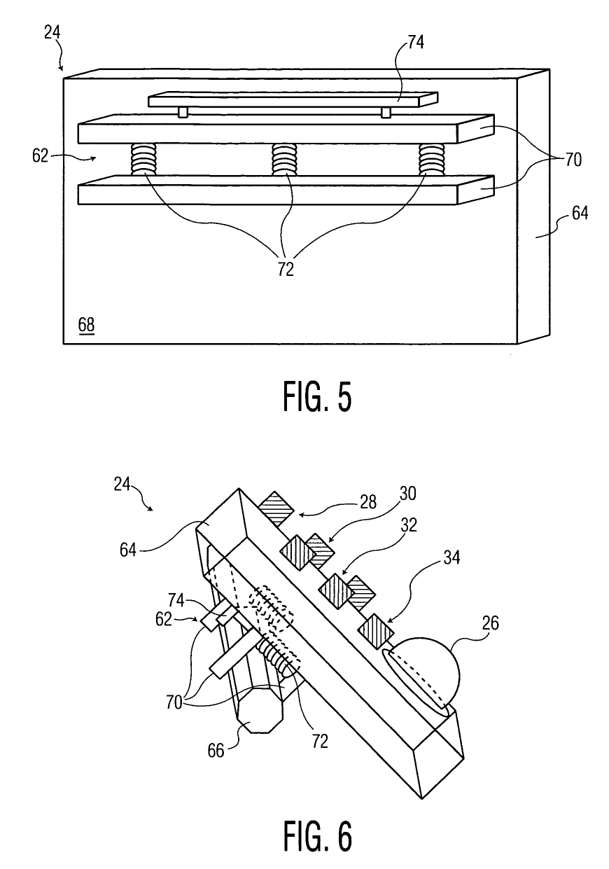

FIG. 5 is a rear view of a remote control device in accordance with the invention showing one attachment mechanism for attaching the remote control device to an object;

FIG. 6 is a side view of the remote control device shown in FIG. 5 when attached to a bed rail; and

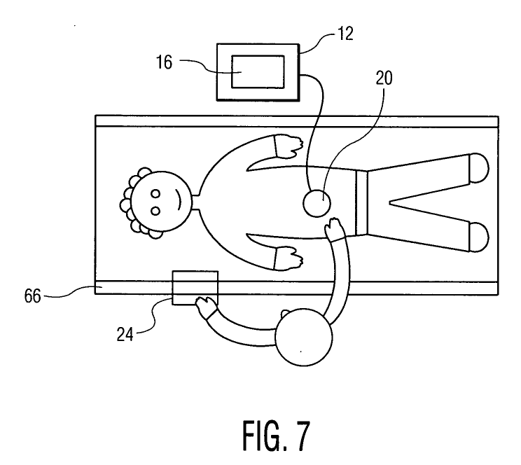

FIG. 7 is a top view of an exemplifying use of the ultrasound machine including a remote control device in accordance with the invention placed in an ergonomically pleasing position.

Referring to the accompanying drawings wherein like reference numerals refer to the same or similar elements, FIG. 1 shows an ultrasound machine 10 in accordance with the invention which includes a housing 12, a user-interface assembly 14 and a screen 16 arranged on the housing 12. The user-interface assembly 14 includes a user-interface input section 18 which includes a keyboard and ultrasound-specific function controls for controlling a transducer 20, i.e., the transmission and reception of ultrasonic waves by the transducer 20, the processing of the ultrasonic waves received by the transducer 20 and the images formed and displayed on the screen 16. Additional function controls may control the storage or recordation of data and images displayed on the screen.

The user-interface input section 18 may include function controls such as a gain control that changes the intensity of the reflected ultrasound image and a freeze control that freezes a displayed ultrasound image on the screen 16. Other controls may include a focus control, a depth control, mode selectors, a trackball or other device for adjusting an indicator on the screen 16. There numerous other controls in the user-interface input section 18 which are included in a standard ultrasound machine as these controls, although used infrequently, are still occasionally needed.

The ultrasound machine 10 also includes a transducer 20 connected to a control unit 22 within the housing 12. The control unit 22 is described in greater detail below.

A remote control device 24 is wirelessly coupled to the control unit 22 within the housing 12 and as shown more clearly in FIG. 2, contains a subset of the function controls of the user-interface input section 18, for example, only the most frequently used function controls. In the non-limiting illustrated embodiment, the remote control device 24 includes an adjustable pointing mechanism such as a trackball 26, a focus adjustment selector 28, a gain adjustment selector 30, a depth adjustment selector 32 and mode selection buttons 34, and optionally TGC's (time gain compensation controls which is a set of gain controls, each for a specific depth in the ultrasound image). Instead of the trackball 26, other adjustable pointing mechanisms may be used such as a pressure-sensitive joystick or a touch pad.

Typically, the remote control device 24 includes at least one control for adjustment of the transmission and reception of the ultrasonic waves by the transducer 20, the processing of the ultrasonic waves by the control unit 22 and the images displayed on the screen 16, e.g., gain, depth, focus and mode selection. An adjustable pointing device for moving an indicator on the screen, such as the trackball 26, is also preferred as it is a frequently-used device.

By providing a subset of only the most frequently used controls for the ultrasound machine on the remote control device 24, the sonographer can more easily control the operation of the ultrasound machine 10, including the image displayed on the screen 16. The sonographer does not have to review an often-crowded user-interface input section 18 to find a particular control but rather can access that control directly on the remote control device 24. Since the remote control device 24 does not have all of the controls found on the user-interface input section 18, it can be formed with more room between the controls resulting in better access and use of the controls. Inadvertent adjustment of an undesired control can thus be substantially avoided or at least substantially curtailed.

The selection of the particular controls to place on the remote control device 24 can be made based on use of the ultrasound machine 10, that is, by determining which controls are used most frequently by the operator. The selection of the particular controls may vary depending on the type of ultrasound machine and its primary use. This is particularly advantageous in connection with the remote control device 24 shown in FIG. 4 wherein the controls on the remote control device 24 can be changed through programming.

Referring to FIG. 2, the remote control device 24 includes a processing unit, represented schematically by box 36 shown in dotted lines, to enable the formation and transmission of wireless (RF) signals based on the manipulation of the controls on the remote control device 24. All of the controls 26, 28, 30, 32 and 34 on the remote control device 24 are thus electrically coupled to the processing unit 36. The signals from the controls 26, 28, 30, 32 and 34 are sent from the processing unit 36 to an antenna 38 which is coupled to the processing unit 36. The processing unit 36 can be similar to the electronic unit used in remote control devices of consumer appliances for cooperating with a housing of the appliance and forming and transmitting signals from control buttons for reception by the appliance. Although shown projecting from the remote control device 24, the antenna 38 may be recessed therein, integrated into the housing of the remote control device or incorporated into the remote control device 24 in any manner known in the remote control art.

For example, sliding of the focus selector 28 on the remote control device 24 will cause the processing unit 36 to form an RF signal indicative of the desired adjustment of the focus of the image being displayed on the screen 18 of the ultrasound machine 10. This signal is transmitted from antenna 38 to antenna 40 coupled to the control unit 22 in the housing 12, as described more fully below. As used herein, the term “coupled to” means directly connected to or indirectly connected to through one or more intermediate components.

The housing 12 includes a mount 42 for receiving and retaining the remote control device 24. As shown in FIG. 1, mount 42 may take the form of a basket 42a (shown in dotted lines) or a slot 42b formed on the front face of the housing 12. Basket 42a and slot 42b are dimensioned to accommodate the remote control device 24 so that the remote control device 24 can be stored together with the housing 12. Various other types of constructions can be employed to retain the remote control device 24 in connection with or on the housing 12.

FIG. 3 is a block diagram of the ultrasonic control unit 22 in the housing 12. The control unit 22 includes beam forming circuitry 44 coupled to the transducer 20 via a cable, a scan converter 46 coupled to the beam forming circuitry 44 and an ultrasound operating system processor 48 coupled to and controlling the beam forming circuitry 44 and the scan converter 46. The beam forming circuitry 44 applies a voltage to the transducer 20 causing it to vibrate and emit ultrasonic energy and also measures the voltages created by the transducer 20 when reflected ultrasonic energy impinges on the transducer 20. The scan converter 46 processes the sensed voltages, usually after amplification, to create an image associated with the reflected signals. This image is displayed on the screen 16 (see FIG. 1).

The user-interface input section 18 is also coupled to the processor 48 to enable processing of the image displayed on the screen 16 by manipulation of the controls in the user-interface input section 18. The user-interface input section 18, the transducer 20, the beam forming circuitry 44, the scan converter 46 and the processor 48 can have any construction known in the art.

The control unit 22 in the housing 12 also includes a signal transmission, reception and processing unit 50 coupled to the antenna 40 for enabling the reception and processing of the signals from the remote control device 24. The signal processing unit 50 can be similar or identical to the electronic unit used in consumer appliances for cooperating with remote control devices. Thus, a communications (RF) link is established between the control unit 22 and the controls 26, 28, 30, 32, 34 on the remote control device 24 via the signal processing unit 50 and the processing unit 36.

The range of transmission of signals between antennas 38 and 40 is limited by the particular type of transmission equipment used in the processing unit 36 and the signal processing unit 50. The range may be about 3 to 5 feet, which is the average length of a conventional cable for a transducer, or greater if so desired.

FIG. 4 shows another embodiment of a remote control device 24 in accordance with the invention. In this embodiment, the remote control device 24 includes a touch screen 52 which can be programmed to display the desired controls. In the non-limiting illustrated embodiment, the touch screen 52 includes a gain region 54 including the notation gain and a slider designed so that application of pressure to the slider and movement thereof (of the pressure site) will result in adjustment of the gain. Similarly, the touch screen 52 includes a focus region 56 and a depth region 58, each with a slider.

The touch screen 52 also includes a pointing region 60 whereby the application and movement of pressure in the pointing region will result in movement of a cursor or indicator on the screen 16. Such a programmable touch screen 52 is commercially available. Programming of the touch screen 52 may be performed using the keyboard portion of the user-interface input section 18 or another input and control accessory.

The remote control device 24 could also include a mini-trackball instead of the pointing region 60. As such, the remote control device 24 would include the mini-trackball on a portion thereof and a programmable touch screen on a remaining portion thereof.

In this embodiment, the processing unit 36 is coupled to the touch screen 52 and the antenna 38 for enabling transmission and reception of signals generated upon touching the touch screen 52 to and from the processing unit 22 in the housing 12.

An advantage of using a programmable touch screen 52 is that the particular controls to be controlled remotely and their location on the touch screen 52 can be changed as needed, e.g., via use of the keyboard portion of the user-interface input section 18 or through other known means. Thus, if it is found that one control is not used very often for a particular type of procedure, that control can be removed when performing the procedure, and possibly replaced by another control used more frequently for that procedure. In this manner, the touch screen 52 can be provided with different controls and/or different locations, sizes and orientations of the controls for different examinations to thereby optimize the control of the ultrasound machine 10.

Moreover, each sonographer may have a preferred arrangement of controls including a particular size, location and orientation of controls. The touch screen 52 can be changed for each sonographer, possibly, by storing the preferred arrangement in memory and accessing the memory before conducting an examination.

In view of the wireless connection between the remote control device 24 and the housing 12 of the ultrasound machine 10, the remote control device 24 can be placed in different positions relative to the patient and preferably in a position and orientation in which it is situated ergonomically for the sonographer, i.e., in a position in which the sonographer is most comfortable manipulating the controls on the remote control device 24. For example, the remote control device 24 can be placed on the patient's bed or on the sonographer's lap.

With reference to FIGS. 5 and 6, the remote control device 24 can be provided with an attachment mechanism 62 for attaching a housing 64 of the remote control device 24 to an object such as a bed rail 66. The attachment mechanism 62 is attached to a rear surface 68 of the housing 64 and includes a pair of clamps 70 coupled to one another by springs 72 and a handle or hand pull 74 for moving one of the clamps 70 toward the other against the bias of the springs 72.

In use, the remote control device 24 is positioned such that the bed rail 66 is between the clamps 70 and then the handle 74 is manipulated to cause the bed rail 66 to be secured between the clamps 70 (see FIG. 6). When used in this manner, the sonographer can position the remote control device 24 on the bed rail 66 and more efficiently conduct the ultrasound examination since one hand can move the transducer 20 on the patient and the remote control device 24 is conveniently placed for easy access by the other hand at a suitable position along the bed rail 66 (for example, in the position shown in FIG. 7).

Other attachment mechanisms can also be used for mounting the remote control device 24 near the patient. For example, an L-shaped mounting bracket could be mounted, upside down, to the rear surface 68 of the remote control device 24 to enable the remote control device 24 to be hung on a footboard, headboard or rail of a bed. Any type of such a hanging mount can be used for a remote control device 24 in accordance with the invention.

The remote control device 24 can also be positioned on a tray bed mount which fits into the I.V. pole openings at the foot of a standard hospital bed. If the tray bed mount includes a special adapter, e.g., for securing a monitor or defibrillator thereto, the housing 64 of the remote control device 24 could be provided with a complementary structure to enable engagement with the adapter (i.e., the same structure as provided on the monitor or defibrillator).

The remote control device 24 can also be positioned on a tilt/swivel top mount for a roll stand. If the tilt/swivel top mount includes a special adapter, e.g., for securing a monitor thereto, the housing 64 of the remote control device 24 could be provided with a complementary structure to enable engagement with the adapter (i.e., the same structure as provided on the monitor).

The remote control device 24 can also be provided with an adapter to enable a hard-wiring connection to the control unit 22 in the housing 12, for example, using a cable. The remote control device 24 could thus include a built-in antenna and transceiver designed for easy switching from a hard-wired to wireless network. When the cable is unplugged, the remote control device 24 would automatically switch to the wireless network.

To supply power to the remote control device 24, a battery compartment may be formed in the housing 64 and circuitry provided for electrically coupling the battery to the components which require electricity to operate, e.g., the processing unit 36. A rechargeable battery could be used in which case, a cord and adapter would be provided in the remote control device 24 to enable recharging of the battery. Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. An ultrasound machine, comprising:
a user-interface assembly including a user-interface input section having a plurality of controls;
an ultrasonic transducer for transmitting and receiving ultrasonic waves;
a screen for displaying an ultrasound image;
a control unit coupled to said user-interface assembly, said screen and said transducer to enable control of said transducer, processing of the received ultrasonic waves and control of the images displayed on said screen via said controls of said user-interface input section; and
a remote control device wirelessly coupled to said control unit and including only a portion of said controls of said user-interface input section to enable remote control of said transducer, of the processing of the received ultrasonic waves and of the images displayed on said screen.

2. The ultrasound machine of claim 1, wherein said portion of said controls on said remote control device includes predetermined, most frequently used controls of said controls on said user-interface input section.

3. The ultrasound machine of claim 1, wherein said portion of said controls on said remote control device includes at least one of a focus adjustment control, a gain adjustment control, a depth adjustment control and a mode selection control.

4. The ultrasound machine of claim 1, wherein said portion of said controls on said remote control device includes a focus adjustment control, a gain adjustment control, a depth adjustment control and mode selection controls.

5. The ultrasound machine of claim 1, wherein said remote control device includes a processing unit coupled to said portion of said controls for forming and transmitting wireless (RF) signals based on manipulation of said portion of said controls.

6. The ultrasound machine of claim 1, wherein said control unit and said remote control device comprises cooperating communication means for enabling transmission and reception of signals at least from said remote control device to said control unit.

7. The ultrasound machine of claim 1, further comprising a housing, said control unit and said user-interface assembly being arranged in connection with said housing, said housing including mounting means for detachably mounting said remote control device on said housing.

8. The ultrasound machine of claim 1, wherein said remote control device includes a touch screen programmable to display said portion of said controls.

9. The ultrasound machine of claim 8, wherein said touch screen includes a pointing region for enabling positional adjustment of an indicator on said screen.

10. The ultrasound machine of claim 8, wherein said remote control device further includes a trackball for enabling positional adjustment of an indicator on said screen.

11. The ultrasound machine of claim 1, wherein said remote control device further includes an adjustable pointing mechanism for enabling positional adjustment of an indicator on said screen.

12. The ultrasound machine of claim 11, wherein said pointing mechanism is a trackball.

13. The ultrasound machine of claim 1, wherein said remote control device includes attachment means for attaching said remote control device to an object.

14. The ultrasound machine of claim 13, wherein said remote control device includes a housing, said attachment means being arranged on a rear surface of said housing.

15. The ultrasound machine of claim 13, wherein attachment means comprises a pair of clamps, springs for coupling said clamps to one another and a handle for moving one of said clamps toward the other of said clamps.

16. A method for enabling ergonomic placement of controls of an ultrasound machine, comprising:
transmitting and receiving ultrasonic waves via an ultrasonic transducer;
processing the received ultrasonic waves to generate an image;
displaying the image generated from the received ultrasonic waves on a screen;
providing a user-interface assembly including a user-interface input section having a plurality of controls for enabling control of the transmission, reception and processing of the ultrasonic waves and images displayed on the screen;
arranging only a portion of the controls of the user-interface input section on a remote control device; and
coupling the remote control device via a wireless connection to a control unit housed in the ultrasonic machine to enable wireless control of the transmission, of the reception and processing of the ultrasonic waves and of the images displayed on the screen via the controls on the remote control device and to enable a sonographer to place the remote control device in an ergonomically pleasing position.

17. The method of claim 16, further comprising selecting the portion of the controls on the remote control device to be predetermined, most frequently used controls.

18. The method of claim 16, wherein the remote control device includes a programmable touch screen, further comprising programming the touch screen to display the portion of the controls.

19. The method of claim 13, further comprising programming the touch screen to display a pointing region for enabling positional adjustment of an indicator on the screen.

20. The method of claim 16, further comprising arranging a trackball on the remote control device for enabling positional adjustment of an indicator on the screen.
